(19) United States
(12) Patent Application Publication
(10) Pub. No.: US 2002/0161013 A1
(43) Pub. Date: Oct. 31, 2002

(54) Method of local anesthesia and analgesia

(75) Inventors: Yuling Liu, Beijing (CN); Wenjuan Yin, Beijing (CN)

Correspondence Address: BIRCH STEWART KOLASCH & BIRCH, PO BOX 747, FALLS CHURCH, VA, 22040-0747, US

(73) Assignee: Wex Medical Intrumentation Co., LTD., Hong Kong (HK)

(21) Appl. No.: 10/006,122

(22) Filed: Dec. 10, 2001

(30) Foreign Application Priority Data
Apr. 26, 2001 (CN) 01110498-8

(51) Int. Cl. A61K 31/519

(52) U.S. Cl. 514/257

(57) ABSTRACT

The present invention relates to a method of obtaining local anesthesia and analgesia to the nerve tissue region of a mammal by administration of an effective dose of sodium channel blocking compounds, including tetrodotoxin and/or saxitoxin and derivatives thereof, in a pharmaceutically suitable vehicle.

### FIELD OF THE INVENTION

The present invention relates to a method of obtaining local anesthesia and analgesia by administration of sodium channel blocking compounds, including tetrodotoxin and/or saxitoxin and derivatives thereof.

### BACKGROUND OF THE INVENTION

According to U.S. patent application Ser. No. (09/702,826), pain is associated with actual or potential injury or tissue damage due to inflammation, ischemia, mechanical or other irritation. Local anesthetics are used to treat pain by blocking neuronal transmission and affect sensation as well as pain. Analgesics are used to relieve pain and they additionally may interfere with the activity of chemical mediators causing inflammation. Under regional anesthesia, patients remain conscious as the systemic physiological activity is exposed to minor interference, and few complications or commemorative signs are observed. Therefore, regional anesthesia is safe and comprehensively utilized in many surgeries.

Regional anaesthesia can be divided into various types, depending on the areas in which the anaesthetic is deposited and the techniques employed. Monheim divided regional anaesthesia into four kinds: (1) Topical (2) Infiltration, (3) Field Block and (4) Nerve Block.

In 1884, William S. Halsted demonstrated that the injection of a nerve trunk in any part of its course is followed by anaesthesia in its entire distribution.

Adams et al U.S. Pat. No. 4,029,793, 1977 states that tetrodotoxin has not found any practical use as an anesthetic. To the opposite, U.S. patent application Ser. Nos. 09/695,053 and 09/702,826 describe that tetrodotoxin can safely be used alone, or in combination with conventional anesthetics, to produce effective anesthesia or analgesia either systemically or locally. Particular examples of inhibiting cancer pain and blocking pain from dental pulp nerve respectively, are described.

In ophthalmic surgeries regional anesthesia is predominantly adopted because it is preferred by the patients and does not cause aftereffects that are commonly seen in systemic anesthesia, such as uneasiness, nausea and vomiting. Ophthalmic regional anesthesia is divided into three kinds:

1. Topical anesthesia: Dripping of a local anesthetic solution directly onto the surface of mucosa to anesthetize the sensory nerve endings. Topical anesthesia is adopted clinically for intraocular pressure measurement, goniometry and suture removal.

2. Infiltration anesthesia: Injection of an anesthetic into a tissue at a subcutaneous or deeper location so as to anesthetize the sensory nerve endings and fibers, such as subconjunctival anesthesia and orbicularis anesthesia.

3. Nerve block: Injection of an anesthetic into or around a nerve trunk so as to produce anesthesia in the distribution of the nerve trunk. Retrobulbar block, peribulbar block and facial nerve block are popular techniques in this category.

Anesthetics currently used, such as lidocaine and bupivacaine, may cause minor lesions, such that regeneration of the cornea epithelium could be inhibited. Current anesthetics also can produce further damage if they are used repeatedly, frequently or chronically. Where longer duration of anesthesia is required, more dosing might be necessary as some current anesthetics only provide brief action.

Therefore, there is a need in the art for methods of producing long lasting local anesthesia and analgesia without significant side effects.

Tetrodotoxin can be used as a local anesthetic and is ten thousand times more powerful than commonly used local non-narcotics, as is discussed by C.Y. Kao and F.A. Fulman, J. Pharmacol., 140, 31-40 (1965). Tetrodotoxin preparations in combination with other widely used anesthetics have been noted in U.S. Pat. No. 4,022,899 and U.S. Pat. No. 4,029,793. According to U.S. Pat. No. 6,030,974, “tetrodotoxin” or “TTX” refers to the amino perhydroquinazoline compounds having the molecular formula C₁₁H₁₇N₃O₈ and to derivatives thereof, including but not limited to anhydrotetrodotoxin, tetrodaminotoxin, methoxytetrodotoxin, ethoxytetrodotoxin, deoxytetrodotoxin and tetrodonic acid (Kao, supra). Examples of TTX analogs include novel TTX analogs isolated from various organisms, as well as those that are partially or totally chemically synthesized. See e.g., Yotsu, M. et al. Agric. Biol. Chem., 53(3):893-895 (1989). Such analogs bind to the same site on the alpha subunit of sodium channels as does TTX.

Adams, et al., U.S. Pat. Nos. 4.022,899 and 4,029,793 describe a local anesthetic composition comprising a mixture in a pharmaceutically acceptable carrier of a particular toxin, namely tetrodotoxin or desoxytetrodotoxin, and another compound, generally a conventional local anesthetic compound or a similar compound having nerve-blocking properties. The conventional local anesthetic can be an aminoacylanilide such as lidocaine, an aminoalkylbenzoate such as procaine, cocaine, an amino carbamate such as diperodon, a N-phenylamidine such as phenacine, a N-aminoalkyl amide such as dibucaine, an aminoketone such as falicain, or an aminoether such as pramoxine.

U.S. Pat. No. 6,030,974 describes a method of producing local anesthesia in a mammal experiencing pain in an epithelial tissue region. The method includes topically administering to the region, in a suitable pharmaceutical vehicle, an effective dose of a longacting sodium channel blocking compound. The sodium channel blocking compound of U.S. Pat. No. 6,030,974 can be a formulation of tetrodotoxin or saxitoxin at a concentration of between 0.001-10 mM. The method described is classified as topical anesthesia in an epithelial tissue region, which is different from the current invention which is a nerve block application. The invention is preferably applied in ophthalmic regional anesthesia. More preferably, the invention relates to retrobulbar injection of a local anesthetic that can block cranial nerve III, IV and V, ciliary ganglion and nerves, thus producing anesthesia in cornea, conjunctiva, iris, ciliary body and choroids, and deep narcosis of the eyeball. Meanwhile, the tension in the lateral rectus can also be reduced so as to decrease the intraocular pressure, allowing for a smooth surgery.

Zapata et al., Pain 72:41-49 (1997) discusses the utilization of tetrodotoxin for the inhibition of neuropathic ectopic activity in neuromas, dorsal root ganglia and dorsal horn neurons. The neuronal activity arises from neuroma caused by mechanical, chemical or ischemic injury. The effect of intravenously administered TTX on the neuronal induction by sciatic nerves in male rats was researched. However, the dosages and effects studied by Zapata et al. were applied to animals under anesthesia and artificial ventilation, thus these doses are above the maximal tolerated dose and the administration was under conditions that are not applicable to the presently intended clinical use of tetrodotoxin.

U.S. patent application Ser. No. 09/702,826 provides a method of producing local analgesia and anesthesia in a mammal experiencing pain in a nerve tissue region. The method includes topically administrating to the region, in a suitable pharmaceutical vehicle, an effective dose of a sodium channel blocking compound, including tetrodotoxin and saxitoxin and their derivatives. A preferred nerve tissue region of that invention is a dental pulp region, a trigeminal nerve region or a sciatic nerve region. The effective dose is administered at a concentration of tetrodotoxin or saxitoxin ranging from 1 mM to 20 mM.

Local administration of any sodium channel blocking compound to the retrobulbar or peribulbar area of the eye is not described in any of the above instances.

### SUMMARY OF THE INVENTION

The present invention offers a solution to the need in the art for methods of producing a nerve block of long duration in ophthalmic surgeries. The inventors have demonstrated for the first time that sodium channel blocking compounds, such as tetrodotoxin and saxitoxin, can generate potent analgesic and anesthesia effects of long duration through retrobulbar or peribulbar block.

The methods and compositions of the invention can be used for relevant ophthalmic surgeries or following injury to the eye. The methods present remarkable advantages, including providing from 30 minutes to 6 hours, preferably under 2 hours, of regional anesthesia and analgesia, without evident side effects.

According to reference [6], conventional synthetic local anesthetics block sodium channels by entering the nerve cell, and then block the channel from the inside. This is in contradistinction to tetrodotoxin, which blocks the sodium channel from the outside of the cell membrane. Synthetic local anesthetics are commonly classified into two major groups based upon whether the intermediate chain is an ester or amide linkage. The main implications of this classification are that the routes of metabolism and the propensity for allergic reactions are predicted by the intermediate chain. The common esters are procaine, chloroprocaine, and tetracaine. The common amides are mepivacaine, prilocaine, lidocaine, bupivacaine and ropivacaine.

The present invention includes methods of producing local anesthesia and analgesia, comprising administering a pharmaceutically acceptable composition of a long-acting sodium channel blocking compound, wherein the compound binds to the extracellular mouth of the sodium channels, to a subject. Preferred compounds include toxins or analogs thereof that specifically bind to a site formed in part by an extracellular region of the alpha subunit of a sodium channel. Most preferred compounds comprise the class of toxins and analogs that specifically bind to a site formed by the SS1 and SS2 extracellular regions of the alpha sub-unit of a sodium channel, wherein such compounds include tetrodotoxin, saxitoxin and analogs thereof.

Accordingly, the present invention is intended to provide a method for producing local analgesia and anesthesia in patients experiencing pain such as that associated with damage to the eye.

One that is knowledgeable in the art will appreciate that retrobulbar or peribulbar injection of a same anesthetic such as the composition described in the current invention will both produce effective nerve block and immobilization.

In one aspect, the invention includes a method of producing local analgesia and anesthesia in the eye and surrounding facial tissues in a subject experiencing pain in the eye.

The method includes topically administering to the retrobulbar or peribulbar area, in a suitable pharmaceutical vehicle, an effective dose of tetrodotoxin or saxitoxin or analogs thereof.

### BRIEF DESCRIPTION OF THE DRAWINGS

FIG. 1 shows a duration of action—dose relationship and an onset time—dose relationship of tetrodotoxin after administering to the retrobulbar region.

### DETAILED DESCRIPTION OF THE INVENTION

As discussed above, the present invention provides a method of producing a local nerve block in the retrobulbar or peribulbar area by administering a anesthetically effective dose of a sodium channel blocking compound, typically tetrodotoxin.

Tetrodotoxin is a nonprotein neurotoxin that is found in multiple diverse animal species, including puffer fish, goby fish, newt, frogs and the blue-ringed octopus.

Saxitoxin is a compound comprising a tetrahydropurine moiety composed of two guanidine units fused together in a stable azaketal linkage, having a molecular formula C₁₀H₁₇N₇O₄. Saxitoxin is commercially available as a hydrochloride salt.

In the examples described herein, tetrodotoxin was administered locally to the retrobulbar or peribulbar area and found to provide potent long-acting pain relief with no signs of systemic or local toxicity. The method of the present invention addresses local analgesia and anesthesia for pain resulting from stimulation to ophthalmic nerves as a result of various ophthalmic diseases and ophthalmic surgery.

For the purpose of this application, a “nerve tissue region” is a portion of tissue that includes an anatomically identifiable nerve. Thus, a nerve tissue region can be tissue that includes the peribulbar or retrobulbar nerves, and the tissue that includes nerves distributed from the peribulbar or retrobulbar nerves. Another example of a nerve tissue region is the tissue through which the cranial nerve III, the cranial nerve IV or the cranial nerve V runs, and tissue that contains nerves that are distributed from these cranial nerves. Another example of a nerve tissue is ciliary ganglion and nerves that distribute therefrom.

Tetrodotoxin useful in the method of the present invention can be obtained from puffer fish organs. A detailed description of production of tetrodotoxin and derivatives thereof is provided in the Chinese patent application no. 00124516.3, filed Sep. 18, 2000 corresponding to co-pending U.S. patent application Ser. No. 09/695,711 filed Oct. 25, 2000 and in Chinese patent application no. 00132673.2 filed Nov. 22, 2000 corresponding to copending U.S. patent application Ser. No. 09/818,863 filed Mar. 28, 2001.

Anesthesia of iris, cornea, conjunctiva, ciliary body and choroids is required in most intraocular surgeries, such as:

extraction of cataract,

intra-ocular implantation of artificial lens,

vitrectomy,

extraction of intra-ocular foreign body,

and operation for retinal detachment.

Regional anesthesia by retrobulbar or peribulbar block is adopted in a majority of those surgeries where it is further required that the lateral rectus be immobilized completely.

Retrobulbar injection of a local anesthetics can block cranial nerve III, IV and V, ciliary ganglion and nerves, producing anesthesia in cornea, conjunctiva, iris, ciliary body and choroids, and deep narcosis of the eyeball. Meanwhile, the tension in the lateral rectus can also be reduced so as to decrease the intraocular pressure, allowing for a smooth surgery.

At present anesthetics used in ophthalmic operations include lidocaine, procaine, bupivacaine and etc. Lidocaine was introduced in 1944 and became a popular anesthetic in various operations. It features a rapid onset about 2 minutes, sound safety, and low possibility of inhibiting the cardiovascular system. It can produce over 20 minutes of duration of action in ophthalmic operations, making it necessary to add more doses where longer operating time is needed. However, an operation has to be discontinued in some cases when lidocaine fails to provide enough duration of action, therefore limiting its use in some types of surgeries.

Bupivacaine, introduced in 1963, is an amide like lidocaine. However, it is much more lipid soluble, conferring a different spectrum of clinical properties. It has a slower onset (10˜15 minutes) and a much longer duration of action than lidocaine, as long as 24 hours when used for peripheral nerve block. Bupivacaine may produce fatal cardiovascular toxicity, central nervous system toxicity, and cardiac arrhythmia. Anxiety, tinnitus, cardiac arrhythmias, coma, convulsions and delayed respiratory arrest may occur when it is administered intravenously. Particularly in ophthalmic applications, while bupivacaine will produce excellent postoperative analgesia, residual diplopia will be experienced by up to 70% of patients next morning. On the other hand, its immobilizing action is inferior to that of other local anesthetics, such as lidocaine.

Tetrodotoxin and Saxitoxin Formulation and Dosages

For use in nerve block, tetrodotoxin and/or saxitoxin is typically administered in an aqueous solution. Typically, the active ingredient tetrodotoxin or saxitoxin is formulated into purified water or a physiological saline solution as a major vehicle. However, it will be appreciated that the clinical formulation can contain other components, including, but not restricted to, buffering means to maintain or adjust pH, such as acetate buffers, citrate buffers, phosphate buffers and borate buffers. Administration of tetrodotoxin has been well studied in a number of animal species. The half-lethal dose for rats by intramuscular injection is between 11 to 18 μg/kg, and that for human is estimated to be about 500 μg/kg.

In one embodiment, the effective dose of tetrodotoxin or saxitoxin is administered from a formulation containing tetrodotoxin or saxitoxin at a concentration of between 0.01 mM to 1 mM (0.0033 to 0.33 mg per ml), preferably 0.03 mM to 0.3 mM (0.01 to 0.1 mg per ml). Up to 10 ml can be administered over a period of 10 to 15 minutes in 3 to 4 doses to obtain a local block. Typically, up to four ml can be injected in the first dose, then 2 to 3 ml can be administered in a second dose. The total amount of TTX administered should not exceed 100 μg. The amount of TTX administered is preferably from 0.5 to 100 μg, more preferably from 10 to 50 μg. Such administration causes a nerve block effect for up to 6 hours, but preferably for 0.25 to 2 hours.

In this application, tetrodotoxin is typically formulated in a vehicle having a pH of between 3.5 to 8.0.

In this application, the pharmaceutical composition does not produce toxic effects or any obvious deleterious side effects.

In the experiments performed in support of the present invention, described above, a 50 μL aliquot of 0.03 mM to 0.3 mM tetrodotoxin was administered by retrobulbar injection to rabbits' eyes. This corresponds to a dose of between 0.5-5 μg of tetrodotoxin. These doses are well below the lethal oral human dose and give a sufficient safety margin to allow for any differences in systemic absorption between local and intramuscular administration.

It will be appreciated that the dosage and concentration of tetrodotoxin or saxitoxin administered is determined on an individual basis, with consideration given to such factors as age and body weight of the patient, as well as to the route of administration and the clinical analgesic and anesthetic requirements. Other compounds useful in the present invention can be administered without the side effects described above for lidocaine and bupivacaine.

### EXAMPLES

The following examples illustrate the methods and compositions of the invention, but are in no way intended to limit the invention.

Animals: New Zealand white rabbits, purchased from the Animal Center of Beijing University, Beijing, P.R.China.

Materials: Tetrodotoxin substance, purity 97.8%, batch no. 000724-1, provided by Nanning Maple Leaf Pharmaceutical Co., Ltd. (Nanning, Guangxi, P.R. China).

1. Preliminary Toxicity Study

Rabbits were randomly divided into 5 groups of 5˜6 each.

Signs of convulsion, rigidity and death were observed at various times after the animals were injected TTX at 250 μg, 125 μg and 25 μg into the retrobulbar area. Those given TTX at 2.5 μg indicated local anesthesia to some extent. One animal in this group demonstrated conjunctival congestion, hydrops (++) and lacrimation in the TTX-treated eye, showed asthma for 1.5 hours and mitigated without intervention. No significant abnormalities were observed in other animals of the same group, neither was local anesthesia observed in the eyes treated with solvent alone.

Based upon the results, the safe dose levels in rabbits were determined to be up to about 2.5 μg.

2. Efficacy study: Local Anesthesia by Retrobulbar Injection of TTX

Method

The experimental animals were randomly divided into groups of 6, half male and half female. The eyelashes of the animals were cut off prior to testing so as to avoid misjudgment due to mistaken contact with the eyelash.

The rabbits were immobilized in special holding boxes for experimental use. To each animal, a 50 μL aliquot of solvent control was injected into the retrobulbar area of the left eye, and a 50 μL aliquot of TTX or lidocaine hydrochloride at various concentrations was injected into the right eye. Corneal sensation was tested with a 5-0 silk suture. The cornea was mechanically stimulated three times (similar to previous rabbit model of corneal anesthesia reported in Maurice D M, Singh T., The absence of corneal toxicity with low-level topical anesthesia. Am J Ophthamol. 99:691-696. (1985)). The rabbit's response was graded in the following fashion: no blink=1; partial blink without full eyelid closure=2; full blink=3. Thus, a score of 3 indicates full responsiveness and a score of 1 indicates full local anesthesia. The highest anesthesia score of the 3 tests was recorded for each time point.

Corneal sensation was tested prior to administration of drugs and again at 1 minute, 2 minutes, 3 minutes, 4 minutes, 5 minutes, 10 minutes, 15 minutes, 1 hour, 2 hours, 3 hours and 10 hours.

Slit lamp biomicroscopy was performed with and without fluorescein stain from impregnated strips moistened with sodium salt solution at 12 and 24 hours after topical administration. The condition of cornea epithelium after dosing was assessed.

Indirect ophthalmoscopy was conducted to assess the condition of the ocular fundus. General examination of the animals was performed within one week after dosing.

Retinal toxicity (or effect on the retinal function) by TTX was evaluated by an electroretinogram at 24 hours after dosing. The method was as follows: complete platycoria was obtained by applying 1% tropine amide, and the eyes were subjected to darkness for 30 minutes. Ketamine at 0.15 mL/kg was injected intramuscularly prior to measurement. Topical anesthesia was obtained by administering 0.4% Beroxil. Skin electrodes were used as the reference and the ground, located subcutaneously in the forehead and the ear root. A cornea contact lens was used as the recording electrode, attached to the center of the cornea with 0.5% methyl fiber. The magnitude of the b wave was determined by the average results of three tests by stimulating with a single flash each time.

The animals were sacrificed by injecting air intravenously one week after dosing. Optical microscopy and transmission electron microscopy were performed subsequently.

Results

Rabbits were randomly divided into 6 groups of 3xx/3xy. Signs of convulsion, rigidity and death were observed in the animals treated with TTX at 10 μg. Animals given TTX at 5 μg, 2.5 μg, 1 μg and 0.5 μg showed local anesthesia in the treated eyes to various extents. While a weak effect was observed with TTX at 0.5 μg, the higher dose of TTX of 5 μg produced a long-lasting local anesthesia of approximately 6 hours.

Ophthalmic and General Examinations

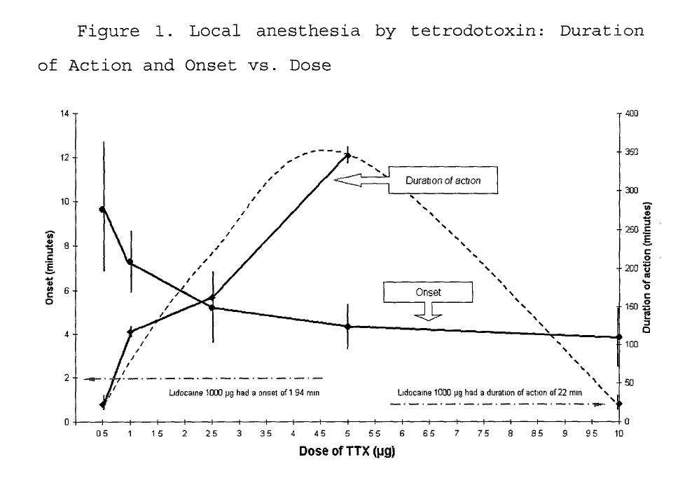

Prior to and at 12 and 24 hours after topical administration, slit lamp biomicroscopy was performed with and without fluorescein stain from impregnated strips moistened with sodium salt solution on the surviving animals. Indirect ophthalmoscopy was also performed. No significant abnormalities were observed. TTX onset and duration of action are shown in FIG. 1.

Electroretinogram (ERG)

Retinal toxicity (or effect on retinal function) by TTX was evaluated with ERG at 24 hours after dosing. No significant differences in the magnitude of ERG b wave were found between the eyes treated with TTX at 2.5 μg or lower doses and their controls. Neither were differences found between those treated with lidocaine at 1000 μg or lower doses and their controls. However, those treated with TTX at 5 μg were observed to have a lower ERG b magnitude than their controls, showing a certain retinal toxicity at this dose level.

General Examination

No changes of food-taking pattern, movement, respiration or alertness were found in the majority of the animals treated with TTX at 5 μg or less. One animal given TTX at 1 μg had a seizure of asthma that lasted 25 minutes and mitigated without intervention. Another animal given TTX at 1 μg appeared paralyzed in the hind limbs the next morning after administration, presumably resulting from improper injection operation as the retrobulbar area of a rabbit approximates the cranial cavity.

Optic and Electron Microscopy

No obvious pathological changes in the retina were found in the animals treated with less than 5 μg TTX during optic and transmission electron microscopy. Of the animals given TTX at 5 μg, the membranes in the outer segments of photoreceptor cells became disorderly and thinned, the formation of mitochondria became disorderly, the number of phagosomes increased in the pigment epithelium cells, the number of outer nuclear layer cells decreased and vacuolar degeneration was observed.

Conclusion:

The test results demonstrated that TTX can produce significant local anesthesia by retrobulbar injection into rabbit eyes. The durations of action were 0.37, 2.0, 2.7 and 5.7 hours when TTX was given at doses of 0.5, 1.0, 2.5 and 5.0 μg, respectively. In the control group, lidocaine at the dose of 1000 μg produced local anesthesia with a duration of action of 0.34 hour. These results evidenced that TTX produces much more potent local anesthesia by retrobulbar injection than does lidocaine, an anesthetic commonly used in current clinical practice.

On the other hand, these studies showed that TTX had a slower onset than lidocaine. While an onset of 1.94 minutes was observed at the dose level of lidocaine of 1000 μg, the onset times for TTX were 9.65, 7.30, 5.19, 4.30 and 3.80 minutes at doses of 0.5, 1.0, 2.5, 5.0 and 10 μg, respectively. This suggests that the onset of TTX is clearly dose-dependent: the onset time decreases when the dose increases.

Retrobulbar injection of TTX at 10 μg and above caused death in rabbits at various times. TTX at 5 μg produced retinal toxicity, whereas TTX at 2.5 μg and under was not observed to produce any toxicity in rabbit eyes. Solvent control (sodium citrate buffer, pH 4.3), even though acidic, did not generate any pronounced stimulation to rabbit eyes.

References

Various articles of the scientific periodical and patent literature are cited herein. Each such article is hereby incorporated by reference in its entirety for all purposes by such citation.

[1] Monheim, L., Local Anesthesia and Pain Control in Dental Practice, 2nd. Edit. C.V. Mosby Co. 1961.

[2] Varvinski et al., Anaesthesia for Opthalmic Surgery Part 1: Regional Techniques, Update in Anesthesia, Issue 6, 1996.

[3] Ritchie J.M., Green N.M., “Local Anesthetics”, pp. 300-302 in Gilman A.G., Goodman L.S., Gilman A., et al. (eds), The Pharmacological Basis of therapeutics, 6^{th} Ed. C. 1980 by Macmillan, New York, NY.

[4] Iliff N.T., Comiplications in Ophthalmic Surgery, pp. 28-34, c. 1983 by Churchill Livingstone Inc., New York, NY.

[5] Hatt Martin, Ophthalmic Plastic and Reconstructive Surgery, c. 1986 by Thieme, New York, NY.

[6] Lawrence Halpern, “Local Anesthetics”, Pharmacology vol. 435, pp. 117-126, Winter Quarter 2000.

We claim:

1. A method of producing local analgesia or anesthesia in a nerve tissue region of a mammal experiencing pain caused by damage to or stimulation of a nerve tissue, comprising locally administering to the nerve tissue region of the mammal an anesthetically or analgesically effective dose of a pharmaceutical composition comprising a compound that binds to the SS1 or SS2 subunit of a sodium channel and a pharmaceutically suitable vehicle;
wherein the nerve tissue region comprises:
(i)the peribulbar nerve and its distribution or a part thereof;
(ii) the retrobulbar nerve and its distribution or a part thereof;
(iii) the whole or a part of cranial nerve III, IV or V and the distribution thereof;
(iv) a ciliary ganglion and the whole or a part of the distribution thereof.

2. The method of claim 1, wherein the method of administration comprises administering the pharmaceutical composition to the intracone space by retrobulbar injection.

3. The method of claim 1, wherein the method of administration is peribulbar injection.

4. The method of claim 1, wherein the compound that binds to the SS1 or SS2 subunit of a sodium channel is tetrodotoxin.

5. The method of claim 4, wherein the effective dose is administered at a concentration of tetrodotoxin of from 0.01 mM to 10 mM.

6. The method of claim 4, wherein the effective dose is administered at a concentration of tetrodotoxin of from 0.03 mM to 3 mM.

7. The method of claim 1, wherein the effective dose of tetrodotoxin can produce local anesthesia or analgesia in the nerve tissue region for a period of 0.5 hour to 6 hours.

8. The method of claim 1, wherein the pharmaceutically suitable vehicle has a pH from 3 to 8.

9. The method of claim 8, wherein the pharmaceutically suitable vehicle has a pH from 4.5 to 7.5.

10. The method of claim 1, wherein the composition further comprises at least one auxiliary acidic solvent selected from dilute acetic acid, dilute hydrochloric acid and dilute citric acid.

11. The method of claim 1, wherein the composition further comprises at least one pH buffer selected from an acetate buffer, a citrate buffer, a phosphate buffer, a borate buffer.

12. The method of claim 1, wherein the composition comprises at least one compound that is tetrodotoxin, anhydrotetrodotoxin, tetrodaminotoxin, methoxytetrodotoxin, ethoxytetrodotoxin, deoxytetrodotoxin or tetrodonic acid.

13. The method of claim 1, wherein the compound that binds to the SS1 or SS2 subunit of a sodium channel is saxitoxin.

14. The method of claim 13, wherein the effective dose is administered at a concentration of saxitoxin of from 0.01 mM to 10 mM.

15. The method of claim 13, wherein the saxitoxin is a compound comprising a tetrahydropurine moiety composed of two guanidine units fused together in a stable azaketal linkage, having a molecular formula C₁₀H₁₇N₇O₄.
